(19) United States
(12) Patent Application Publication
(10) Pub. No.: US 2023/0212670 A1
(43) Pub. Date: Jul. 6, 2023

(54) RAPID VIRAL NUCLEIC ACID DETECTION KIT PREPARED BY USING NOVEL RECA ENZYME AND DETECTION METHOD THEREOF

(71) Applicant: ZHEJIANG SHANCE HEQISHI BIO-SCI & TECH. CO., LTD., Jiaxing (CN)

(72) Inventors: Qi CHENG, Jiaxing (CN); Jinhui WANG, Jiaxing (CN); Dongjie HU, Jiaxing (CN); Jun CHEN, Jiaxing (CN); Zixin DENG, Jiaxing (CN)

(21) Appl. No.: 17/851,843

(22) Filed: Jun. 28, 2022

(30) Foreign Application Priority Data
Jan. 6, 2022 (CN) 202210007779.4

(51) Int. Cl. C12Q 1/6876; C12Q 1/6844; C12N 9/48

(52) U.S. Cl. CPC C12Q 1/6876; C12Q 1/6844; C12N 9/48; C12Q 2600/16

(57) ABSTRACT

The present invention provides a rapid viral nucleic acid detection kit prepared by using a novel RecA enzyme and the detection method thereof. By editing the recombinase RecA gene, the expressed RecA protein has better solubility and recombinase activity. The RecA protein is used to prepare recombinase dry powder, further the formula of the recombinase dry powder and the ratio are optimized; and specific primers for the ASFV p72 gene are designed for the rapid nucleic acid detection of ASFV, significantly improving the detection sensitivity. In addition, the detection time is short, which effectively avoids missed detection and false detection, and helps prevention of epidemic.

### SEQUENCE LISTING

A sequence listing is being submitted with this application. This sequence listing is submitted as file name “2022-06-28-Repl-Sequence-Listing” with a file size of 6,886 bytes and a date of creation of Jun. 28, 2022. This document is hereby incorporated by reference in its entirety.

### FIELD OF THE INVENTION

The present invention belongs to the technical field of molecular diagnostic biology, relates to a rapid viral nucleic acid detection kit prepared by using a novel RecA enzyme and a detection method thereof, more particularly relates to a rapid nucleic acid detection kit for African swine fever virus and a detection method thereof.

### BACKGROUND OF THE INVENTION

African swine fever (ASF) was first reported in Kenya in 1921, and has been present in sub-Saharan African countries. ASF spread to Western Europe and Latin American countries in 1957, and was eliminated in most countries in time; however, it is still epidemic in Portugal, southwestern Spain and Sardinia in Italy. Since 2007, ASF has occurred, spread and prevalent in many countries around the world. In August 2018, it spread to Shenyang, China. So far, ASF cases have been found and reported in most regions in China, and emergency culling and harmless treatment have been carried out, to avoid the further spread of the epidemic.

ASF is an acute, febrile, highly contagious zoonotic disease caused by African swine fever virus (ASFV) in domestic pigs and wild boars. Pigs of all breeds and ages can be infected, and the morbidity and mortality can reach 100%. The disease has the characteristics of easy infection, short onset process, rapid spread, difficult control and high fatality rate. The World Organization for Animal Health has listed ASF as a statutory reportable animal disease, and in China, it is classified as a first-class animal disease. Once it occurs, it will cause large-scale death of pigs on the farms, bringing huge losses to the health of pig herds and the profitability of farms. Due to the lack of effective immunization vaccines and therapeutic drugs, only the prevention and control of ASF is implemented, usually by culling and pollution-free treatment measures, which has a serious impact on the economic incomes of local farmers and herdsmen.

African swine fever virus (ASFV) is a DNA virus. The main detection methods are enzyme-linked immunosorbent assay (ELISA), polymer chain reaction assay (PCR), real-time fluorescence-based quantitative PCR, and colloidal gold test paper detection methods. The colloidal gold test paper detection method is a novel immunodetection technology for the specific detection of antibody antigens by using colloidal gold as a tracer marker and chromatographic technology. It has the advantages of rapidity, sensitivity and specificity, but it is prone to have false positive results. The PCR method has high accuracy and can be effectively applied to detection, but the amplification time is long, electrophoresis detection is required, the throughput is low, and the operation is complicated. The real-time fluorescence-based quantitative PCR method is to quantitatively detect the target gene according to the fluorescent signals, which has a high sensitivity and a strong specificity, and has gradually become an important method for clinical diagnosis, quarantine, and food safety inspection of ASFV. However, the method has high requirements for technicians and takes 1.5 to 2 hours; and the detection equipment is expensive, which is not conducive to promotion in pig farms and remote areas.

The emergence of in vitro isothermal nucleic acid amplification technology has expanded the application scope of PCR technology once again and made it more convenient to operate. Existing methods of in vitro isothermal nucleic acid amplification mainly include loop-mediated isothermal amplification (LAMP), recombinase polymerase amplification (RPA) and recombinase-aid amplification (RAA).

CN110699490B provides a RAA isothermal fluorescence detection method of ASFV CD2V gene, but its sensitivity is low due to the low recombinase mediation efficiency in the process of RAA amplification. The template amount of 100 copies/μL and 2 μL is equivalent to a sensitivity of 100*2=200 copies/test, the detection time is long, and the amplification time is 20 to 30 minutes. When the viral burden is low, false negative results are prone to occur, resulting in missed detection and false detection.

Therefore, there is an urgent need for a rapid nucleic acid detection method with higher sensitivity, more convenience and specificity, especially a rapid nucleic acid detection method for ASFV, to avoid the possibility of missed and false detections and help prevent the spread of the epidemic.

### SUMMARY OF THE INVENTION

In order to solve the above problems, the present invention provides a rapid viral nucleic acid detection kit prepared by using a novel RecA enzyme and a detection method thereof. By editing the recombinase RecA gene, the expressed RecA protein has better solubility and recombinase activity. The RecA protein is used to prepare recombinase dry powder, further the formula of the recombinase dry powder and the ratio are optimized; and specific primers for the ASFV p72 gene are designed for the rapid nucleic acid detection of ASFV, significantly improving the detection sensitivity. In addition, the detection time is short, and a sample of 5 copies/μL can be detected to the minimum. After calculation, the sensitivity is about 50 copies/test, and the detection time is only 5 to 20 minutes, which effectively avoids missed detection and false detection, and solves the problems of low sensitivity, false positive result, complicated operation, time-consuming, expensive detection instruments, etc. in the existing ASF detection products or methods. Furthermore, the dependence on instruments is low. It is not only suitable for the PCR systems currently used in the market, but also for the isothermal PCR systems; meanwhile, it is suitable for the rural areas or remote farms, and has a good application prospect.

The recombinase amplification used in the present invention is a recombinase-aid amplification and is a rapid isothermal nucleic acid amplification technology. The recombinase obtained from bacteria or fungi is utilized, and under room temperature, the recombinase can tightly bind to the primer, to form a polymer of the enzyme and the primer. When the primer searches for a completely matched complementary sequence on the template DNA, with the aid of the single-stranded DNA binding protein, the double-stranded structure of the template DNA is opened, and a new DNA complementary strand is formed under the action of DNA polymerase, then the amplified product grows exponentially. By using the fluorescent probe labeling and exonuclease digestion, the quantitative analysis of results can be performed in a timely manner.

In one aspect, the present invention provides a recombinase RecA gene, and the RecA gene has a sequence described in the Sequence Listing SEQ ID NO: 6.

RecA protein is a homologous recombinant protein derived from Deinococcus radiodurans. Deinococcus radiodurans has extreme radiation resistance and efficient DNA damage repair mechanism, which can quickly repair various DNA damage. RecA protein is one of the important DNA repair proteins for homologous recombination repair of DNA. The RecA protein derived from Deinococcus radiodurans has strong resistance to stress, but for the expressed wild-type RecA protein cloned from the genomes of Deinococcus radiodurans, the product in the E. coli expression system is an inclusion body, and the in vitro enzyme activity is low.

In the present invention, gene editing is performed on the basis of the wild-type RecA gene (SEQ ID NO: 5), and a part of the structural domain of the T4 bacteriophage recombinant protein is introduced into the RecA protein, to obtain a novel RecA gene (SEQ ID NO: 6). The RecA protein obtained by expressing the novel RecA gene has significantly improved solubility and recombinase activity. When the RecA protein obtained by expressing the novel RecA gene is used for recombinase-aid amplification to detect viral nucleic acid, the detection sensitivity can be significantly improved and the detection time can be shortened.

In another aspect, the present invention provides a recombinase dry powder comprising the RecA protein expressed by the RecA gene as described above.

Further, the recombinase dry powder also comprises uvsY protein and ssb protein (single-chain binding protein).

Studies have shown that uvsY protein can assist RecA protein and improve the mediation efficiency of RecA protein, thereby improving the detection sensitivity of nucleic acid detection.

RecA protein, uvsY protein and ssb protein together mediate the melting of the template and achieve the efficiency of strand replacement between the template and the primer.

Further, the recombinase dry powder also comprises Exo protein.

The Exo protein can recognize the tetrahydrofuran bond of the probe, cleave the probe, release fluorescence, and perform fluorescence detection.

Further, the recombinase dry powder also comprises DNA polymerase P.

DNA polymerase P is derived from DNA polymerase I of Escherichia coli, and can specifically extend DNA after strand replacement.

Further, the recombinase dry powder also comprises 20 ng/μL-50 ng/μL of RecA protein, 10 ng/μL-50 ng/μL of uvsY protein, 10 ng/μL-50 ng/μL of DNA polymerase P, 30 ng/μL-100 ng/μL of ssb protein, and 20 ng/μL-60 ng/μL of Exo protein.

The ratio and concentration of various proteins in the recombinase dry powder are critical to the sensitivity and specificity of the reaction, especially the RecA, UvsY and ssb proteins that mediate the melting of the template and achieve the chain replacement between the template and the primer.

Further, the recombinase dry powder comprises 30 ng/μL of RecA protein, 20 ng/μL of uvsY protein, 30 ng/μL of DNA polymerase P, 50 ng/μL of ssb protein, and 20 ng/μL of Exo protein.

In still another aspect, the present invention provides a rapid viral nucleic acid detection kit, comprising the recombinase dry powder as described above.

Further, the kit also comprises a primer-probe set, a reaction Buffer, Buffer B, a negative control and a positive control.

The reaction Buffer can provide salt ions and stable pH for the reaction, which affects the activity of the enzyme; the recombinase dry powder is the freeze-dried powder of a plurality of enzymes, which is added to the reaction solution to amplify the target fragment at 25-42° C.; the primer-probe set is related to the reaction sensitivity and specificity; Buffer B is a reaction activator.

It can be apprehended that the kit provided by the present invention can be used for the detection of any viral nucleic acid. As long as the nucleotide sequence of the virus to be tested is provided, rapid detection can be performed by the kit provided by the present invention, and the detection sensitivity can be improved and the detection time can be shortened.

Further, the virus is ASFV, and the primer-probe set comprises:

(1) An upstream primer, whose sequence is shown in SEQ ID NO: 1 in the Sequence Listing;

(2) A downstream primer, whose sequence is shown in SEQ ID NO: 2 in the Sequence Listing;

(3) A fluorescent probe sequence, whose sequence is shown in SEQ ID NO: 3 in the Sequence Listing.

Further, the primer-probe set comprises primer probes: 0.5-2 μL of 10 mM upstream primer ASF-FP, 0.5-2 μL of 10 mM downstream primer ASF-RP, and 0.5-1 μL of 10 mM probe ASF-P.

Further, the reaction Buffer is: 10-30 mM Tris-HCl (pH7.5), 0.1 mM-0.5 mM dNTP, PEG8000 or other molecular-weight PEG with a mass percent of 3%-9%, 1-5 mM DTT, 10-20 mM ATP, 10-50 mM phosphoenolpyruvate, and 500-1500 ng/μL pyruvate kinase.

Further, the Buffer B is 2-10 mM Mg2+; the positive control is pseudovirus containing ASFV (ASF) p72 gene fragment; the negative control is normal saline.

The ASFV p72 gene sequence is shown in SEQ ID NO: 4 in the Sequence Listing.

In still another aspect, the present invention provides a method for rapid detection of viral nucleic acid using the above-mentioned kit, comprising the following steps:

(1) extracting nucleic acid to be tested, preparing a positive control and a negative control;

(2) preparing recombinase dry powder and placing into a reaction tube, adding a reaction Buffer, an upstream primer, a downstream primer, and a fluorescent probe;

(3) adding the nucleic acid to be tested, the positive control and the negative control to the reaction tube, then adding Buffer B, shaking and mixing well, and centrifuging;

(4) performing isothermal amplification and fluorescence analysis of the sample to be tested.

In some embodiments, the virus is ASFV.

In some embodiments, the nucleic acid is the ASFV p72 gene.

The recombinase amplification rapid virus detection method provided by the present invention is to carry out the reaction under the condition of 25° C.-42° C., the dependence on the instrument is low, the reaction time is short, the detection time only needs 5-20 minutes; with the characteristics of high sensitivity, strong specificity, short detection time, simple operation and low costs for instruments.

In some embodiments, the present invention utilizes isothermal amplification technology to amplify and detect ASFV nucleic acid at a temperature of 37-42° C.

In some embodiments, the reaction procedure for recombinase-aid amplification is as follows:

| | | | Number of | Collection |
|---|---|---|---|---|
| Step | Temperature | Time/cycle | [symbol] | of [symbol] |
| Preheating | 37-42° C. | 40 s | 1 | Yes |
| Amplificatio [symbol] | 37-42° C. | 30 s | 40 | Not [symbol] |

[symbol]

indicates data missing or illegible when filed

Remarks: During the amplification process, high-concentration samples only need 10-20 cycles to amplify the curve, while low-concentration samples need 30-40 cycles to amplify the curve.

Further, the present invention provides a method for rapid detection of ASF nucleic acid, comprising the following steps:

\textcircled{1} extracting nucleic acids according to the instructions of the viral nucleic acid extraction kit, and extracting the positive and negative controls simultaneously;

\textcircled{2} adding 10 μL of reaction Buffer, 0.5-2 μL of 10 mM upstream primer, 0.5-2 μL of 10 mM downstream primer, and 0.5-1 μL of 10 mM probe into an dry enzyme powder tube; preparing the dry enzyme powder: adding 50 μL of liquid enzyme, and then performing freeze-drying into solid powder, wherein the content of each protein is the concentration*50 μL;

\textcircled{3} Adding 5-20 μL of nucleic acid extraction product to the reaction tube, then adding 1-3 μL of Buffer B, supplementing the reaction system with ddH₂O to 50 μL, shaking and mixing well, and centrifuging quickly for 5-10 S;

\textcircled{4} Placing the reaction tube into the PCR system, reacting at 25-42° C. for 5-20 minutes, collecting the fluorescent signal once every 30 s; after the detection, judging whether the sample is a positive sample according to the fluorescent signals.

The present invention has the following beneficial effects:

1. A novel RecA gene is obtained by gene editing of the wild-type RecA gene. The RecA protein obtained by expressing the novel RecA gene has significantly improved solubility and recombinase activity; when the novel RecA protein is used for viral nucleic acid detection, it can significantly improve the detection sensitivity and shorten the detection time;

2. The novel RecA protein is used together with uvsY protein and ssb protein to prepare recombinase dry powder, which further improves the recombinase-mediated efficiency, thereby improving the sensitivity of nucleic acid detection and further shortening the detection time;

3. The primer-probe set that is most suitable for the detection of ASF p72 gene using the recombinase dry powder is found, and the reaction system is optimized, so that a minimum sample of 5 copies/μL can be detected. After calculation, the sensitivity is about 50 copies/test, and the detection time only takes 5-20 minutes, thus, the detection speed is fast, the sensitivity is high, and the specificity is strong.

4. The operation is simple and the adaptability of the instruments is extensive. It can not only adapt to the current PCR systems available on the markets, but also to an isothermal amplification instrument. It does not rely on precise nucleic acid amplification instruments. The detection time is short and the sensitivity is high. It can be manually operated or used in an automated platform, is widely suitable for customers' fast, effective and economical testing needs. The dry powder enzyme system can be transported at room temperature, adapt to many scenarios, and has broad application prospects.

### BRIEF DESCRIPTION OF THE DRAWINGS

FIGS. 1 to 3 show the detection results of 3 groups of primer-probe sets in the Example 3, wherein FIG. 1 is the detection result of primer-probe set 1, FIG. 2 is the detection result of primer-probe set 2, and FIG. 3 is the detection result of primer-probe set 3;

FIGS. 4 to 6 show the detection results of the two groups of recombinase systems for pseudoviruses with different concentrations in the Example 6, wherein FIG. 4 is the detection result of 2×104 copies/μL pseudoviruses, FIG. 5 is the detection result of 2×103 copies/μL pseudoviruses, and FIG. 6 is the detection result of 200 copies/μL pseudoviruses;

FIGS. 7 to 9 show the detection results of pseudoviruses with different concentrations in the Example 7, wherein FIG. 7 is the detection results of samples with high concentrations (2×104 copies/μL, 2×103 copies/μL), FIG. 8 is the detection results of samples with medium concentrations (200 copies/μL, 100 copies/μL, 50 copies/μL), and FIG. 9 is the detection results of samples with low concentrations (50 copies/μL, 20 copies/μL and 5 copies/μL).

### DETAILED DESCRIPTION OF THE EMBODIMENTS

The present invention will be further described in detail below with reference to the embodiments. It should be noted that, the following embodiments are intended to facilitate the understanding of the present invention, but do not constitute any limitation on the present invention. All reagents that are not specified in the embodiments are all known products and are commercially available.

### Example 1 Editing of the RecA Gene Provided by the Present Invention

This example was directed to the RecA gene (Gene ID: 68370659, SEQ ID NO: 5) of wild type Deinococcus radiodurans, and the gene sequence was as follows:

ATGAGCAAGGACGCCACCAAAGAAATCTCCGCCCCCACCGACGCCAAGG
AACGCAGCAAGGCCATCGAAACAGCCATGAGCCAGATCGAAAAGGCCTT
CGGCAAGGGCTCGATCATGAAACTGGGCGCCGAGAGCAAACTCGACGTG
CAGGTCGTCAGCACCGGCAGCCTCAGCCTTGACCTCGCACTGGGCGTGG
GCGGCATTCCGCGTGGCCGCATCACCGAGATCTACGGCCCCGAGTCGGG
CGGCAAGACCACCCTGGCCCTCGCCATCGTCGCGCAGGCCCAGAAAGCG
GGCGGCACCTGTGCGTTTATCGACGCCGAGCACGCGCTCGACCCGGTGT
ACGCCCGCGCCCTGGGCGTGAACACCGACGAACTGCTGGTGTCGCAGCC
CGACAACGGCGAGCAGGCGCTCGAAATCATGGAACTGCTGGTGCGTTCG
GGCGCGATTGATGTGGTGGTCGTGGACTCGGTGGCTGCTCTGACCCCCC
GCGCCGAAATCGAGGGCGACATGGGCGACAGCCTGCCCGGTCTTCAGGC
CCGCCTGATGTCGCAGGCGCTGCGCAAGCTGACGGCGATTCTCTCCAAG
ACCGGCACCGCCGCCATCTTCATCAACCAGGTTCGCGAGAAAATCGGCG
TGATGTACGGCAACCCCGAAACCACCACCGGGGGCCGGGCGCTGAAGTT
CTACGCCAGCGTGCGCCTCGACGTGCGTAAGATCGGCCAGCCCACCAAG
GTCGGCAACGACGCGGTCGCCAACACCGTCAAGATCAAGACCGTGAAGA
ACAAGGTCGCCGCCCCCTTCAAGGAAGTCGAACTCGCGCTGGTCTACGG
CAAGGGCTTCGACCAGCTCAGCGACCTCGTGGGCCTGGCCGCCGACATG
GACATCATCAAGAAGGCCGGCAGCTTCTACTCCTACGGCGACGAGCGCA
TCGGCCAGGGCAAGGAAAAGACCATCGCCTACATCGCCGAGCGCCCCGA
GATGGAGCAGGAAATCCGCGACCGCGTGATGGCCGCCATCCGCGCGGGC
AACGCGGGCGAAGCACCGGCCCTGGCCCCCGCGCCTGCCGCGCCCGAAG
CCGCCGAAGCGTAA

The gene editing was performed on the wild type RecA gene. A partial domain of the T4 phage recombinant protein was introduced into the RecA protein; by inserting the sequence “AAAAATGGCTGGTATGCTCGTGAATTTCTTGACGAAGAAACTGGCGAGATGATTCGC GAAGAAAAATCTTGGCGTGCAAAAGATACTAACTGCACTACATTCTGG” (SEQ ID NO: 13) after the wild type “GGACATC”, the novel RecA gene (SEQ ID NO: 6) was obtained, as follows:

ATGAGCAAGGACGCCACCAAAGAAATCTCCGCCCCCACCGACGCCAAGG
AACGCAGCAAGGCCATCGAAACAGCCATGAGCCAGATCGAAAAGGCCTT
CGGCAAGGGCTCGATCATGAAACTGGGCGCCGAGAGCAAACTCGACGTG
CAGGTCGTCAGCACCGGCAGCCTCAGCCTTGACCTCGCACTGGGCGTGG
GCGGCATTCCGCGTGGCCGCATCACCGAGATCTACGGCCCCGAGTCGGG
CGGCAAGACCACCCTGGCCCTCGCCATCGTCGCGCAGGCCCAGAAAGCG
GGCGGCACCTGTGCGTTTATCGACGCCGAGCACGCGCTCGACCCGGTGT
ACGCCCGCGCCCTGGGCGTGAACACCGACGAACTGCTGGTGTCGCAGCC
CGACAACGGCGAGCAGGCGCTCGAAATCATGGAACTGCTGGTGCGTTCG
GGCGCGATTGATGTGGTGGTCGTGGACTCGGTGGCTGCTCTGACCCCCC
GCGCCGAAATCGAGGGCGACATGGGCGACAGCCTGCCCGGTCTTCAGGC
CCGCCTGATGTCGCAGGCGCTGCGCAAGCTGACGGCGATTCTCTCCAAG
ACCGGCACCGCCGCCATCTTCATCAACCAGGTTCGCGAGAAAATCGGCG
TGATGTACGGCAACCCCGAAACCACCACCGGGGGCCGGGCGCTGAAGTT
CTACGCCAGCGTGCGCCTCGACGTGCGTAAGATCGGCCAGCCCACCAAG
GTCGGCAACGACGCGGTCGCCAACACCGTCAAGATCAAGACCGTGAAGA
ACAAGGTCGCCGCCCCCTTCAAGGAAGTCGAACTCGCGCTGGTCTACGG
CAAGGGCTTCGACCAGCTCAGCGACCTCGTGGGCCTGGCCGCCGACATG
GACATCAAAAATGGCTGGTATGCTCGTGAATTTCTTGACGAAGAAACTG
GCGAGATGATTCGCGAAGAAAAATCTTGGCGTGCAAAAGATACTAACTG
CACTACATTCTGGATCAAGAAGGCCGGCAGCTTCTACTCCTACGGCGAC
GAGCGCATCGGCCAGGGCAAGGAAAAGACCATCGCCTACATCGCCGAGC
GCCCCGAGATGGAGCAGGAAATCCGCGACCGCGTGATGGCCGCCATCCG
CGCGGGCAACGCGGGCGAAGCACCGGCCCTGGCCCCCGCGCCTGCCGCG
CCCGAAGCCGCCGAAGCGTAA

Shanghai Sangon Biotech was entrusted to perform gene synthesis. A novel RecA protein was obtained by expressing the novel RecA gene in E. coli. The specific expression method and purification were obtained by traditional methods. The protein amino acid sequencing analysis showed that the expressed recombinant protein was normally translated by the nucleotide sequence.

Example 2 the Rapid Viral Nucleic Acid Detection Kit Provided in the Present Invention and Use Thereof in Virus Detection

The components of the detection kit prepared with the novel RecA protein provided by Example 1 were as follows:

1. Enzyme dry powder tube: 1.0 μg-2.5 μg (equivalent to 20 ng/μL-50 ng/μL) of the novel RecA protein (Hangzhou Heqishi, lot: A20211011), 0.5 μg-2.5 μg (equivalent to 10 ng/μL-50 ng/μL) of uvsY (Hangzhou Heqishi, lot: Y20111011), 0.5 μg-2.5 μg (equivalent to 10 ng/μL-50 ng/μL) of DNA polymerase P (Hangzhou Heqishi, lot: P20211014) and 1.5 μg-5 μg (equivalent to 30 ng/μL-100 ng/μL) of ssb protein (Hangzhou Heqishi, lot: S20210910), 1.0 m-3.0 m (equivalent to 20 ng/μL-60 ng/μL) of Exo protein (Hangzhou Heqishi, lot: E20210911).

2. Reaction Buffer: 10-30 mM Tris-HCl (pH7.5), 0.1 mM-0.5 mM dNTP, PEG8000 or PEG of other molecular weights at a mass percent of 3%-9%, 1-5 mM DTT, 10-20 mM ATP, 10-50 mM phosphoenolpyruvate, 500-1500 ng/μl pyruvate kinase;

3. Activator (Buffer B): 2-10 mM Mg2+;

4. Primer-probe set: For the virus to be tested, the corresponding upstream primers, downstream primers and probes were designed, and the ratio was as follows: 0.5-2 μL of 10 mM upstream primer, 0.5-2 μL of 10 mM downstream primer, 0.5-1 μL of 10 mM probe;

5. Positive control: pseudovirus containing the gene segment of the virus to be tested;

6. Negative control: normal saline.

The kit provided in this example could be used for the detection of any virus, and the detection method was as follows:

(1) Extract the nucleic acid to be tested, and prepare positive and negative controls;

(2) Add a reaction Buffer, an upstream primer, a downstream primer and a fluorescent probe to an enzyme dry powder tube;

(3) Add the nucleic acid to be tested, positive control and negative control to the reaction tube, then add Buffer B, shake and mix well, and centrifuge;

(4) Perform isothermal amplification and fluorescence analysis of the sample to be tested.

Due to limited space, only ASFV was used as an example for illustration in this example.

The primer-probe set designed for the p72 gene of ASFV included an upstream primer ASF-FP (SEQ ID NO: 1), a downstream primer ASF-RP (SEQ ID NO: 2) and a probe ASF-P (SEQ ID NO: 3).

The specific components of the rapid detection kit for ASFV nucleic acid were as follows:

1. Enzyme dry powder tube: 1.0 m-2.5 μg (equivalent to 20 ng/μL-50 ng/μL) of the novel RecA protein (Hangzhou Heqishi, lot: A20211011), 0.5 μg-2.5 μg (equivalent to 10 ng/μL-50 ng/μL) of uvsY (Hangzhou Heqishi, lot: Y20111011), 0.5 m-2.5 μg (equivalent to 10 ng/μL-50 ng/μL) of DNA polymerase P (Hangzhou Heqishi, lot: P20211014) and 1.5 m-5 μg (equivalent to 30 ng/μL-100 ng/μL) ssb protein (Hangzhou Heqishi, lot: S20210910), 1.0 m-3.0 m (equivalent to 20 ng/μL-60 ng/μL) of Exo protein (Hangzhou Heqishi, lot: E20210911).

2. Reaction Buffer: 10-30 mM Tris-HCl (pH7.5), 0.1 mM-0.5 mM dNTP, PEG8000 or PEG of other molecular weights at a mass percent of 3%-9%, 1-5 mM DTT, 10-20 mM ATP, 10-50 mM phosphoenolpyruvate, 500-1500 ng/μl pyruvate kinase;

3. Activator (Buffer B): 2-10 mM Mg²⁺;

4. Primer-probe set: 0.5-24, of 10 mM upstream primer ASF-FP (SEQ ID NO: 1), 0.5-24, of 10 mM downstream primer ASF-RP (SEQ ID NO: 2), 0.5-14, of 10 mM probe ASF-P (SEQ ID NO: 3);

5. Positive control: pseudovirus containing ASFV p72 gene fragment, as shown in SEQ ID NO: 4;

6. Negative control: normal saline.

The method for rapid detection of ASFV using the rapid detection kit of ASFV nucleic acid comprised the following steps:

(1) extract nucleic acids according to the instructions of the viral nucleic acid extraction kit, and extract the positive and negative controls simultaneously;

(2) add 10 μL of reaction Buffer, 1 μL of 10 mM upstream primer, 1 μL of 10 mM downstream primer, and 0.5 μL of 10 mM probe into an dry enzyme powder tube;

(3) add 5 μL of nucleic acid extraction product, 5 μL of positive control and 5 μL of negative control to the reaction tube, then add 2.5 μL of Buffer B, supplement the reaction system with 30 μL of ddH₂O to 50 μL, shake and mix well, and centrifuge quickly for 5-10S;

(4) Place the reaction tube into the PCR system, react at 25-42° C. for 5-20 minutes, collect the fluorescent signal once every 30 s; after the detection, judge whether the sample was a positive sample according to the fluorescent signals.

The reaction program of isothermal amplification was shown in Table 1:

TABLE 1
Reaction program of isothermal amplification

| | | | Number of | Collection |
|---|---|---|---|---|
| Step | Temperature | Time/cycle | cycles | of [symbol] |
| Preheating | 37-42° C. | 40 s | 1 | [symbol] |
| Amplificati [symbol] | 37-42° C. | 30 s | 40 | [symbol] |

[symbol]

indicates data missing or illegible when filed

Remarks: During the amplification process, high-concentration samples only needed 10-20 cycles to amplify the curve, while low-concentration samples needed 30-40 cycles to amplify the curve.

### Example 3 Screening of Primer-Probe Set for Detection of ASFV

Unlike conventional PCR, which uses the complementary binding of primers and templates in the annealing process, the isothermal nucleic acid technology utilizes the polymer of recombinase and primers to search for the complementary sequences on the template DNA that completely match with them under normal temperature conditions; then with the aid of the single-stranded DNA binding protein, the double-stranded structure of the template DNA is opened, and a new DNA complementary strand is formed under the action of DNA polymerase P. Since the principle of isothermal amplification technology is different from that of conventional PCR, the design of the primer is also completely different. The length of the primer is longer, about 30 nt, to ensure the identification and specificity of the chain during the amplification process. Because the primer is longer, it is easier to form secondary structures, which is amplified with the primer dimer at 37-42° C. The secondary structure is not easy to open, which will affect the amplification efficiency, so the design is more difficult.

At present, there is no design software for primer probes for recombinase-aid amplification, and extensive screening is required. The probe design of recombinase-aid amplification is unique. The fluorescent group and the quenching group are modified inside the sequence, and are connected by a tetrahydrofuran bond. The exonuclease recognizes the tetrahydrofuran bond, cuts the probe and releases fluorescence, and the 3′ end of the probe is phosphorylated to block.

In this example, the kit and detection method provided by Example 2 were used to detect ASFV, wherein the recombinase dry powder was composed of 20 ng/μL of novel RecA protein, 10 ng/μL of uvsY, 30 ng/μL of DNA polymerase P and 30 ng/μL of ssb protein, 20 ng/μL Exo protein; and multiple groups of primer-probe sets were designed for the p72 gene of ASFV. Three groups were selected and listed in Table 2, namely, group 1, group 2, and group 3. Synthesis was performed in Shanghai Sangon Biotech and the primers and probes were diluted to 10 mM according to the dilution method.

TABLE 2
Primer-probe set designed for ASFV p72 gene

| Group | Name | Sequence |
|---|---|---|
| Group | ASF-FP | CAGATATAGATGAACATGCGTCTGGAAG (SEQ ID NO: 1) |
| 1 | ASF-RP | ATCCTCATCAACACCGAGATTGGCACA (SEQ ID NO: 2) |
| | ASF-P | TATCTCTGCGTGGTGAGTGGGCTGCA/I6FAMdT/A/idSP// |
| | | IBHQ1dT/GGCGTTAACAACAT (SEQ ID NO: 3) |
| Group | ASF-FP1 | TGTGCCAATCTCGGTGTTGATGAGGAT (SEQ ID NO: 7) |
| 2 | ASF-RP1 | CCACACCAACAATAACCACCACGATG (SEQ ID NO: 8) |
| | ASF-P1 | GTTCCAGGTAGGTTTTAATCCTATAAACA/I6FAMdT/A/idSP/A/ |
| | | IBHQ1dT/TCAATGGGCCAT (SEQ ID NO: 9) |
| Group | ASF-FP2 | CCGAACTTGTGCCAATCTCGGTGTTGATG (SEQ ID NO: 10) |
| 3 | ASF-RP2 | AACGCAGGTGACCCACACCAACAATAACC (SEQ ID NO: 11) |
| | ASF-P2 | TAGGTTTTAATCCTATAAACATATAT/I6FAMdT/C/idSP/A/ |
| | | IBHQ1dT/GGGCCATTTAAGAGC (SEQ ID NO: 12) |

The synthesized pseudovirus was diluted with physiological saline to different concentration gradients (0, 50, 100, 200 copies/μL), and then extracted with a purchased viral nucleic acid extraction kit [Tiangen Biotech (Beijing) Co., Ltd., viral genomic DNA/RNA extraction kit (DP315)]. According to the instructions, 200 μL of the sample was taken for nucleic acid extraction, then eluted with 100 μL of RNase-Free ddH2O, and then 5 μL of the eluted product was taken for rapid nucleic acid detection.

Reaction System:

| | | |
|---|---|---|
| Reaction Buffer | 10 | μL |
| DNA | 5.0 | μL |
| Activator | 2.5 | μL |
| Upstream primer | 1 | μL |
| Downstream primer | 1 | μL |
| Probe | 0.5 | μL |
| ddH2O | 30 | μL |
| Total | 50.0 | μL |

### Reaction Program of Isothermal Amplification:

| | | | Number of | Collection of |
|---|---|---|---|---|
| Step | Temperature | Time/cycle | cycles | fluorescence |
| Preheating | 37-42° C. | 40 s | 1 | Yes |
| Amplificati [symbol] | 37-42° C. | 30 s | 40 | Not [symbol] |

[symbol]

indicates data missing or illegible when filed

Remarks: During the amplification process, high-concentration samples only needed 10-20 cycles to amplify the curve, and low-concentration samples needed 30-40 cycles to amplify the curve.

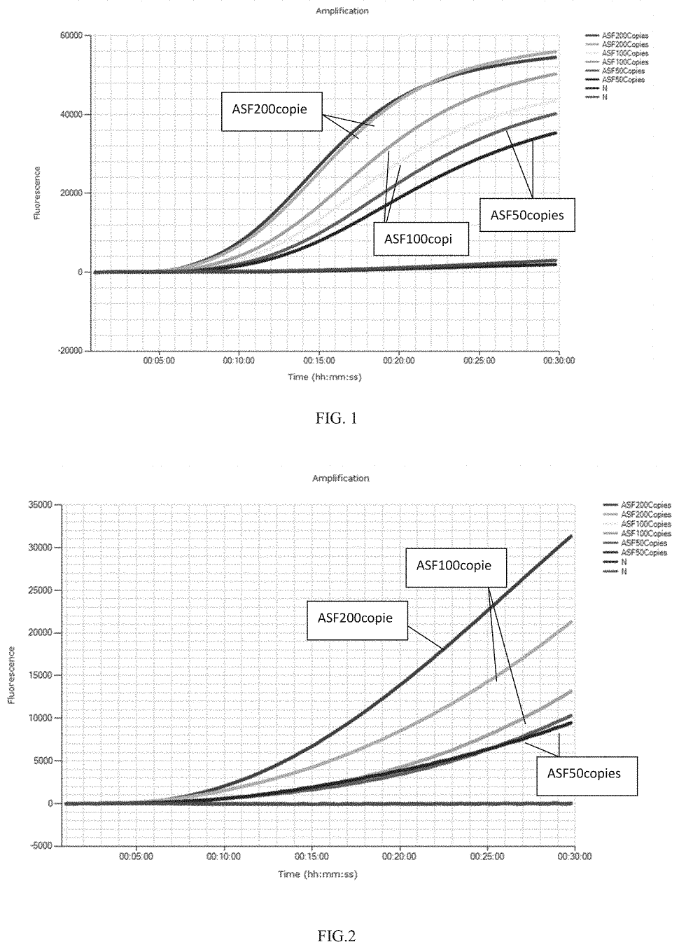

The test results were shown in FIGS. 1 to 3 respectively, wherein FIG. 1 was the detection result of the primer-probe set 1, FIG. 2 was the detection result of the primer-probe set 2, and FIG. 3 was the detection result of the primer-probe set 3.

According to the results in FIGS. 1 to 3, the primer probe of group 1 had an obviously high sensitivity and a better amplification curve (S-shaped), and a higher fluorescence intensity (ordinate value) when detected. When the sample concentration was as low as 50 copies/μL, it could still be accurately detected. So, the primer-probe set of the kit was preferably group 1.

### Example 4 Influence of Novel RecA Protein and Existing RecA Protein on ASFV Detection

In this example, the kit and detection method provided by Example 2 were used to detect the ASFV p72 gene. The recombinase dry powder was composed of 30 ng/μL of RecA protein, 20 ng/μL of uvsY, 30 ng/μL of DNA polymerase P, 30 ng/μL of ssb protein, 50 ng/μL of Exo protein; wherein the RecA protein used the novel RecA protein provided by Example 1 and the existing RecA protein (Hangzhou Heqishi, lot: R20210912). The synthesized pseudovirus was diluted with normal saline to 0, 5, 20, 50, 100, 150, 200 copies/μL for detection. The detection results were shown in Table 3.

TABLE 3
Influence of novel RecA protein and existing
RecA protein on ASFV detection

| Recombinase dry powder | Sensitivity (copies/μL) | Detection time |
|---|---|---|
| Novel RecA protein | 5 | 5-20 min |
| Existing RecA protein | 150 | 20-30 min |

As shown in Table 3, the novel RecA protein expressed by the novel RecA gene could significantly improve the detection sensitivity of the ASFV p72 gene, so that the detection sensitivity reached 5 copies/μL, and the detection time was shortened to 5-20 min.

### Example 5 Effect of Adding uvsY Protein on ASFV Detection

In this example, the kit and detection method provided by Example 2 were used to detect the ASFV p72 gene. The recombinase dry powder was composed of 30 ng/μL of RecA protein, 20 ng/μL of uvsY, 30 ng/μL of DNA polymerase P and 50 ng/μL of ssb protein, 20 ng/μL of Exo protein. Wherein, the uvsY protein was added or not added to the recombinase dry powder, respectively, to investigate whether the uvsY protein had the effect of auxiliary-mediated amplification on RecA protein. The synthesized pseudovirus was diluted with normal saline to 0, 5, 20, 50, 100, 150, 200 copies/μL for detection. The detection results were shown in Table 4.

TABLE 4
Influence of the novel RecA protein and
existing RecA protein on ASFV detection

| Recombinase dry powder | Sensitivity (copies/μL) | Detection time |
|---|---|---|
| uvsY-containing protein | 5 | 5-20 min |
| uvsY-free protein | 50 | 10-20 min |

As shown in Table 4, the uvsY protein had a very significant auxiliary effect on the novel RecA protein. The addition of the uvsY protein could significantly improve the detection sensitivity of the ASFV p72 gene and further shorten the detection time. The possible reason was that a partial domain of the T4 phage recombinant protein was introduced to the novel RecA protein, and the uvsY protein could assist the RecA protein to bind to the target fragment; therefore, the uvsY protein could assist the novel RecA protein and increase the recombinase-mediated efficiency, thereby improving the amplification efficiency, shortening the amplification time and improving the detection sensitivity.

The detection time was also closely related to the virus concentration of the sample to be tested. When the concentration was low, the amplification time would be longer. However, when the virus concentration of the sample to be tested was the same, the addition of uvsY protein could further shorten the amplification time, thereby shortening the detection time.

### Example 6 Optimization of Recombinase Amplification System

The reaction Buffer is used to maintain the pH value of the reaction system, provide raw materials and energy for DNA synthesis. Buffer B is the activator of the reaction, and an appropriate amount of Buffer B is added to start the reaction. The ssb protein is a single-chain binding protein, and the RecA protein is a homologous recombination protein, when cloned and expressed in Deinococcus radiodurans, Deinococcus radiodurans has extreme radiation resistance and an efficient DNA damage repair mechanism, which can quickly repair various DNA damages. RecA protein is one of the important DNA repair proteins, which performs homologous recombination repair of DNA. The RecA protein derived from Deinococcus radiodurans has strong stress resistance, but for the wild-type RecA protein cloned from the genome of Deinococcus radiodurans, the product in the E. coli expression system is an inclusion body with low in vitro enzyme activity. Gene editing (SeQ.NO6) was performed on the basis of the wild-type recA gene (SEQ.NO5), which improved the solubility of the expressed product and the recombinase activity. The melting of templates mediated by the novel RecA protein, Uvs Y and ssb proteins and the strand replacement between the template and the primer require ATP for energy. DNA polymerase P is derived from DNA polymerase I of Escherichia coli and can specifically extend the DNA after strand replacement. The Exo protein recognizes the tetrahydrofuran bond of the probe, cuts the probe, releases fluorescence, and performs fluorescence detection.

In this example, the kit and detection method provided by Example 2 were used to detect the ASFV p72 gene, and it was found that the ratio and concentration of various proteins in the system were critical to the sensitivity and specificity of the reaction, especially the RecA, UvsY and ssb proteins that mediated the melting of the template and achieved the chain replacement between the template and the primer. In this example, on the basis of the existing reaction system, further optimization was carried out to increase the contents of ssb protein, novel RecA protein and UvsY protein, and obviously improve the detection sensitivity.

Preparation of Recombinase Dry Powder:

Group 1: 20 ng/μL novel RecA protein, 10 ng/μL uvsY protein, 30 ng/μL DNA polymerase P and 30 ng/μL ssb protein, 20 ng/μL Exo protein;

Group 2: 30 ng/μL novel RecA protein, 20 ng/μL uvsY protein, 30 ng/μL DNA polymerase P and 50 ng/μL ssb protein, 20 ng/μL Exo protein.

The synthesized pseudovirus was diluted with physiological saline to different concentration gradients (20000, 2000, 200 copies/μL), and then extracted with a purchased viral nucleic acid extraction kit [Tiangen Biotech (Beijing) Co., Ltd., viral genomic DNA/RNA extraction kit (DP315)]. According to the instructions, 200 μL of the sample was taken for nucleic acid extraction, then eluted with 100 μL of RNase-Free ddH2O, and then 5 μL of the eluted product was taken for rapid nucleic acid detection. The reaction solution system and the reaction procedure were shown in Example 3.

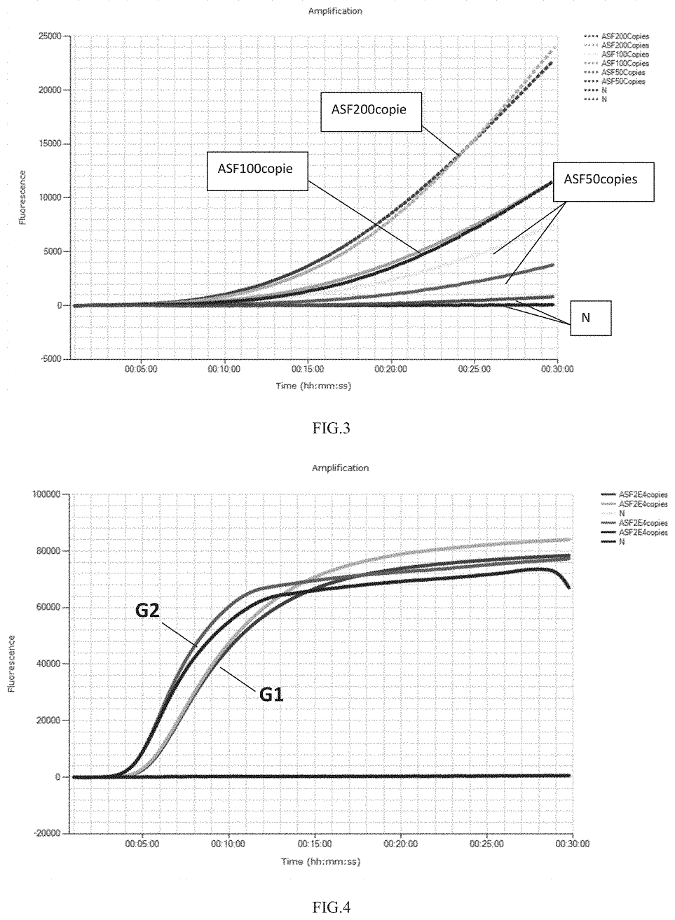

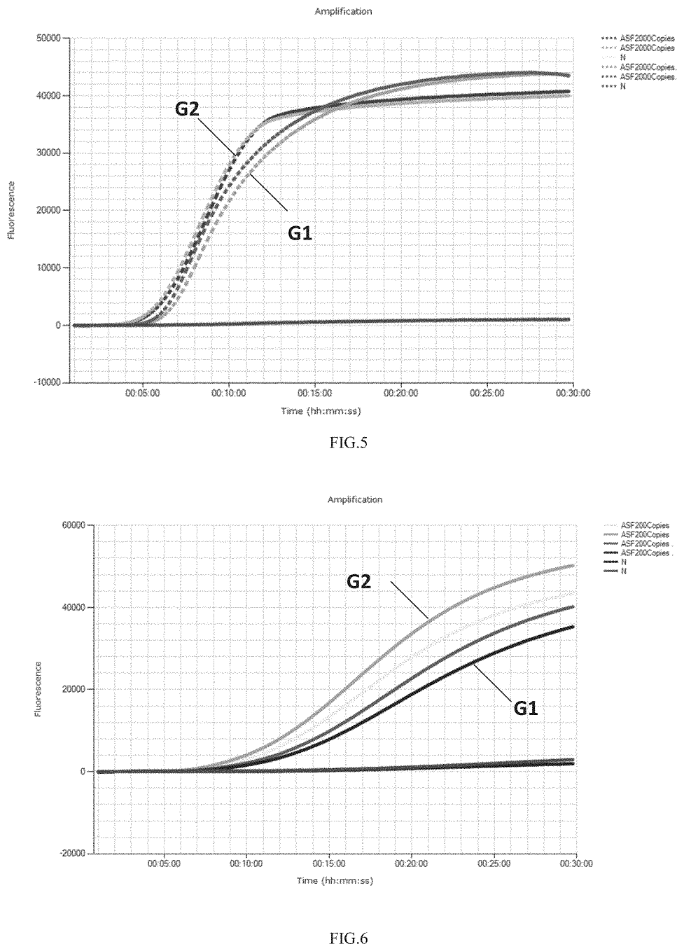

The detection results were shown in FIGS. 4 to 6, wherein FIG. 4 was the detection result of 2×104 copies/μL pseudoviruses, FIG. 5 was the detection result of 2×103 copies/μL pseudoviruses, and FIG. 6 was the detection result of 200 copies/μL pseudoviruses; as shown in FIGS. 4 to 6, regardless of the virus content of 200 copies/μL, 2000 copies/μL or 2×104 copies/μL, when the recombinase dry powder system of group 2 was used, the peak would be apparently earlier than that of group 1, and there was a more obvious exponential phase and plateau phase, the amplification effect was better, the sensitivity was high, and the appearance time was faster. Therefore, the recombinase dry powder system of group 2 was preferably used, namely, 30 ng/μL of novel RecA protein, 20 ng/μL of uvsY protein, 30 ng/μL of DNA polymerase P, 50 ng/μL of ssb protein, and 20 ng/μL of Exo protein.

### Example 7 Influence of Different Concentrations of Gradient Samples on Detection

In this example, the kit and detection method provided by Example 2 were used to detect the ASFV p72 gene. The recombinase dry powder was composed of 30 ng/μL of RecA protein, 20 ng/μL of uvsY, 30 ng/μL of DNA polymerase P and 50 ng/μL of ssb protein, and 20 ng/μL of Exo protein. The synthesized pseudovirus was diluted with physiological saline to different concentration gradients (2×104 copies/μL, 2×103 copies/μL, 200 copies/μL, 100 copies/μL, 50 copies/μL, 20 copies/μL, 5 copies/μL), and then extracted with a purchased viral nucleic acid extraction kit [Tiangen Biotech (Beijing) Co., Ltd., viral genomic DNA/RNA extraction kit (DP315)]. According to the instructions, 200 μL of the sample was taken for nucleic acid extraction, then eluted with 100 μL of RNase-Free ddH2O, and then 5 μL of the eluted product was taken for rapid nucleic acid detection. The reaction solution system and reaction procedure were shown in Example 3.

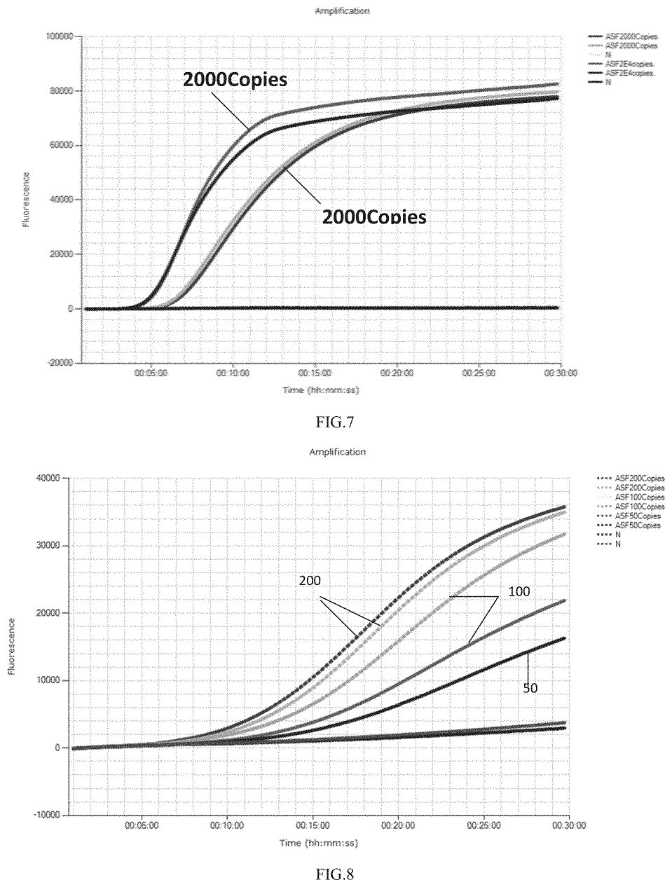

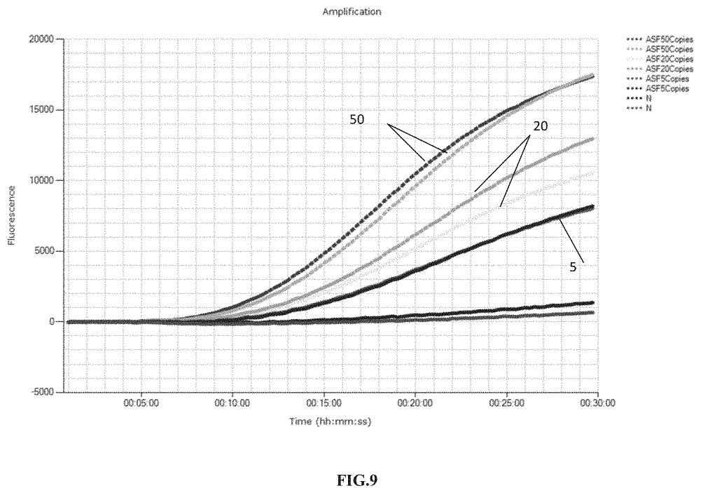

The test results were shown in FIGS. 7 to 9, wherein FIG. 7 was the detection results of samples with high concentrations (2×104 copies/μL, 2×103 copies/μL), FIG. 8 was the detection results of samples with medium concentrations (200 copies/μL, 100 copies/μL, 50 copies/μL), and FIG. 9 was the detection results of samples with low concentrations (50 copies/μL, 20 copies/μL and 5 copies/μL).

As shown in FIGS. 7 to 9, under the detection condition of high concentration samples, the fluorescence signal appeared in 2 to 5 minutes; under the detection condition of low concentration samples, the fluorescence signal appeared in 5 to 10 minutes, and reached the plateau phase in 20 minutes. A sample of at least 5 copies/μL could be detected, and the calculated sensitivity was about 50 copies/test (sensitivity=[5 copies/ul×200 ul (sample)/100]×5 ul). The sample detection speed was fast and the sensitivity was high.

Although the present invention has been described in detail above with general illustrations and specific embodiments, the foregoing embodiments are exemplary, and the specific features, structures, materials, or characteristics may be combined and incorporated in any suitable manner in any one or more embodiments. It is obvious to those skilled in the art that some modifications or improvements can be made on the basis of the present invention. Therefore, these modifications, improvements, substitutions or variations made without departing from the spirit of the present invention shall fall within the scope of protection of the present invention.

Sequence Listing

SEQ ID NO: 1
ASF-FP:
CAGATATAGATGAACATGCGTCTGGAAG
SEQ ID NO: 2
ASF-RP:
ATCCTCATCAACACCGAGATTGGCACA
SEQ ID NO: 3
ASF-P:
TATCTCTGCGTGGTGAGTGGGCTGCA/I6FAMdT/A/idSP//IBHQ1dT/GGCGTTAACAACAT
SEQ ID NO: 4
P72:
AGGTAATGTGATCGGATACGTAACGGGGCTAATATCAGATATAGATGAACATGCGTCTG
GAAGAGCTGTATCTCTATCCTGAAAGCTTATCTCTGCGTGGTGAGTGGGCTGCATAAT
GGCGTTAACAACATGTCCGAACTTGTGCCAATCTCGGTGTTGATGAGGATTTTGATCG
GAGATGTTCCAGGTAGGTTTTAATCCTATAAACATATATTCAATGGGCCATTTAAGAGC
AGACATTAGTTTTTCATCGTGGTGGTTATTGTTGGTGTGGGTCACCTGCGTTTTATGGA
CACGTATCAGCGAAAAGCGAACGCGTTTTACAAAAAGGTTGTGTATTTCAGGGGTTA
CAAACAGGTTAT
SEQ ID NO: 5
Wild type RecA:
ATGAGCAAGGACGCCACCAAAGAAATCTCCGCCCCCACCGACGCCAAGGAACGCAG
CAAGGCCATCGAAACAGCCATGAGCCAGATCGAAAAGGCCTTCGGCAAGGGCTCGA
TCATGAAACTGGGCGCCGAGAGCAAACTCGACGTGCAGGTCGTCAGCACCGGCAGC
CTCAGCCTTGACCTCGCACTGGGCGTGGGCGGCATTCCGCGTGGCCGCATCACCGAG
ATCTACGGCCCCGAGTCGGGCGGCAAGACCACCCTGGCCCTCGCCATCGTCGCGCAG
GCCCAGAAAGCGGGCGGCACCTGTGCGTTTATCGACGCCGAGCACGCGCTCGACCC
GGTGTACGCCCGCGCCCTGGGCGTGAACACCGACGAACTGCTGGTGTCGCAGCCCG
ACAACGGCGAGCAGGCGCTCGAAATCATGGAACTGCTGGTGCGTTCGGGCGCGATTG
ATGTGGTGGTCGTGGACTCGGTGGCTGCTCTGACCCCCCGCGCCGAAATCGAGGGCG
ACATGGGCGACAGCCTGCCCGGTCTTCAGGCCCGCCTGATGTCGCAGGCGCTGCGCA
AGCTGACGGCGATTCTCTCCAAGACCGGCACCGCCGCCATCTTCATCAACCAGGTTC
GCGAGAAAATCGGCGTGATGTACGGCAACCCCGAAACCACCACCGGGGGCCGGGCG
CTGAAGTTCTACGCCAGCGTGCGCCTCGACGTGCGTAAGATCGGCCAGCCCACCAAG
GTCGGCAACGACGCGGTCGCCAACACCGTCAAGATCAAGACCGTGAAGAACAAGGT
CGCCGCCCCCTTCAAGGAAGTCGAACTCGCGCTGGTCTACGGCAAGGGCTTCGACCA
GCTCAGCGACCTCGTGGGCCTGGCCGCCGACATGGACATCATCAAGAAGGCCGGCA
GCTTCTACTCCTACGGCGACGAGCGCATCGGCCAGGGCAAGGAAAAGACCATCGCCT
ACATCGCCGAGCGCCCCGAGATGGAGCAGGAAATCCGCGACCGCGTGATGGCCGCC
ATCCGCGCGGGCAACGCGGGCGAAGCACCGGCCCTGGCCCCCGCGCCTGCCGCGCC
CGAAGCCGCCGAAGCGTAA
SEQ ID NO: 6
Novel typeRecA:
ATGAGCAAGGACGCCACCAAAGAAATCTCCGCCCCCACCGACGCCAAGGAACGCAG
CAAGGCCATCGAAACAGCCATGAGCCAGATCGAAAAGGCCTTCGGCAAGGGCTCGA
TCATGAAACTGGGCGCCGAGAGCAAACTCGACGTGCAGGTCGTCAGCACCGGCAGC
CTCAGCCTTGACCTCGCACTGGGCGTGGGCGGCATTCCGCGTGGCCGCATCACCGAG
ATCTACGGCCCCGAGTCGGGCGGCAAGACCACCCTGGCCCTCGCCATCGTCGCGCA
GGCCCAGAAAGCGGGCGGCACCTGTGCGTTTATCGACGCCGAGCACGCGCTCGACC
CGGTGTACGCCCGCGCCCTGGGCGTGAACACCGACGAACTGCTGGTGTCGCAGCCC
GACAACGGCGAGCAGGCGCTCGAAATCATGGAACTGCTGGTGCGTTCGGGCGCGAT
TGATGTGGTGGTCGTGGACTCGGTGGCTGCTCTGACCCCCCGCGCCGAAATCGAGGG
CGACATGGGCGACAGCCTGCCCGGTCTTCAGGCCCGCCTGATGTCGCAGGCGCTGC
GCAAGCTGACGGCGATTCTCTCCAAGACCGGCACCGCCGCCATCTTCATCAACCAGG
TTCGCGAGAAAATCGGCGTGATGTACGGCAACCCCGAAACCACCACCGGGGGCCGG
GCGCTGAAGTTCTACGCCAGCGTGCGCCTCGACGTGCGTAAGATCGGCCAGCCCAC
CAAGGTCGGCAACGACGCGGTCGCCAACACCGTCAAGATCAAGACCGTGAAGAACA
AGGTCGCCGCCCCCTTCAAGGAAGTCGAACTCGCGCTGGTCTACGGCAAGGGCTTCG
ACCAGCTCAGCGACCTCGTGGGCCTGGCCGCCGACATGGACATCAAAAATGGCTGGT
ATGCTCGTGAATTTCTTGACGAAGAAACTGGCGAGATGATTCGCGAAGAAAAATCTT
GGCGTGCAAAAGATACTAACTGCACTACATTCTGGATCAAGAAGGCCGGCAGCTTCT
ACTCCTACGGCGACGAGCGCATCGGCCAGGGCAAGGAAAAGACCATCGCCTACATC
GCCGAGCGCCCCGAGATGGAGCAGGAAATCCGCGACCGCGTGATGGCCGCCATCCG
CGCGGGCAACGCGGGCGAAGCACCGGCCCTGGCCCCCGCGCCTGCCGCGCCCGAAG
CCGCCGAAGCGTAA
SEQ ID NO: 7
ASF-FP1:
TGTGCCAATCTCGGTGTTGATGAGGAT
SEQ ID NO: 8
ASF-RP1:
CCACACCAACAATAACCACCACGATG
SEQ ID NO: 9
ASF-P1:
GTTCCAGGTAGGTTTTAATCCTATAAACA/I6FAMdT/A/idSP/A/IBHQ1dT/TCAATGGGCC
AT
SEQ ID NO: 10
ASF-FP2:
CCGAACTTGTGCCAATCTCGGTGTTGATG
SEQ ID NO: 11
ASF-RP2:
AACGCAGGTGACCCACACCAACAATAACC
SEQ ID NO: 12
ASF-P2:
TAGGTTTTAATCCTATAAACATATAT/I6FAMdT/C/idSP/A/IBHQ1dT/GGGCCATTTAAGAG
C
SEQ ID NO: 13
AAAAATGGCTGGTATGCTCGTGAATTTCTTGACGAAGAAACTGGCGAGATGATTCGC
GAAGAAAAATCTTGGCGTGCAAAAGATACTAACTGCACTACATTCTGG

1. A nucleotide sequence encoding a recombinase RecA, wherein the nucleotide sequence is a sequence of SEQ ID NO: 6.

2. The nucleotide sequence according to claim 1, wherein the nucleotide sequence of the partial structure encoding T4 bacteriophage protein is inserted after GACATC of the sequence shown in SEQ ID NO: 5 in the Sequence Listing, and the nucleotide sequence SEQ ID NO: 5 is a partial sequence of the wild type Deinococcus radiodurans.

3. The nucleotide sequence according to claim 2, wherein the nucleotide sequence encoding the T4 phage protein is shown in SEQ ID NO: 13.

4. A recombinase dry powder, comprising the RecA protein encoded by the RecA gene described in claim 1.

5. The recombinase dry powder according to claim 4, further comprising uvsY protein, ssb protein and Exo protein.

6. The recombinase dry powder according to claim 5, wherein the recombinase dry powder comprises 20 ng/μL-50 ng/μL of RecA protein, 10 ng/μL-50 ng/μL of usvY protein, 10 ng/μL-50 ng/μL of DNA polymerase P, 30 ng/μL-100 ng/μL of ssb protein, 20 ng/μL-60 ng/μL of Exo protein.

7. A rapid viral nucleic acid detection kit, comprising the recombinase dry powder as described in claim 4.

8. The kit according to claim 7, wherein the kit further comprises a primer-probe set, a reaction Buffer, Buffer B, a negative control and a positive control.

9. The kit according to claim 8, wherein the virus is ASFV, and the primer-probe set comprises: an upstream primer, whose sequence is shown in SEQ ID NO: 1 in the Sequence Listing; a downstream primer, whose sequence is shown in SEQ ID NO: 2 in the Sequence Listing; and a fluorescent probe sequence whose sequence is shown in SEQ ID NO: 3 in the Sequence Listing.

10. The kit according to claim 9, wherein the reaction Buffer is 10-30 mM Tris-HCl (pH7.5), 0.1 mM-0.5 mM dNTP, PEG8000 or PEG of other molecular weights at a mass percent of 3%-9%, 1-5 mM DTT, 10-20 mM ATP, 10-50 mM phosphoenolpyruvate, and 500-1500 ng/μL pyruvate kinase.

11. The kit according to claim 8, wherein the Buffer B is 2-10 mM Mg2+; the positive control is a pseudovirus containing the ASFV p72 gene fragment; and the negative control is normal saline.

12. The kit according to claim 8, wherein the concentration of the novel RecA protein is 20 ng/μL, the concentration of the uvsY protein is 10 ng/μL, the concentration of the DNA polymerase P is 30 ng/μL, the concentration of the ssb protein is 30 ng/μL, and the concentration of the Exo Protein is 20 ng/μL.

13. A method for detecting ASFV, comprising the following steps:
extracting nucleic acid to be tested, preparing a positive control and a negative control;
preparing recombinase dry powder and placing into a reaction tube, adding a reaction Buffer, an upstream primer, a downstream primer, and a fluorescent probe;
adding the nucleic acid to be tested, the positive control and the negative control to the reaction tube, then adding Buffer B, shaking and mixing well, and centrifuging;
performing isothermal amplification and fluorescence analysis of the sample to be tested;
wherein the recombinase dry powder comprises a novel RecA protein encoded by a nucleotide sequence, and the nucleotide sequence is the sequence described in SEQ ID NO: 6.

14. The method according to claim 13, wherein the recombinase dry powder further comprises uvsY protein, ssb protein and Exo protein.

15. The method according to claim 13, wherein the recombinase dry powder comprises 20 ng/μL-50 ng/μL of RecA protein, 10 ng/μL-50 ng/μL of usvY protein, 10 ng/μL-50 ng/μL of DNA polymerase P, 30 ng/μL-100 ng/μL of ssb protein, 20 ng/μL-60 ng/μL of Exo protein.

16. The method according to claim 13, wherein the nucleotide sequence of the partial structure encoding T4 bacteriophage protein is inserted after GACATC of the sequence shown in SEQ ID NO: 5 in the Sequence Listing, and the nucleotide sequence SEQ ID NO: 5 is a partial sequence of the wild type Deinococcus radiodurans.

17. The method according to claim 13, wherein the nucleotide sequence encoding the T4 bacteriophage protein is shown in SEQ ID NO: 13.

18. The method according to claim 13, wherein the primer-probe set comprises: an upstream primer, whose sequence is shown in SEQ ID NO: 1 in the Sequence Listing; a downstream primer, whose sequence is shown in SEQ ID NO: 2 in the Sequence Listing; and a fluorescent probe sequence whose sequence is shown in SEQ ID NO: 3 in the Sequence Listing.

19. The method according to claim 13, wherein the concentration of the novel RecA protein is 20 ng/μL, the concentration of the uvsY protein is 10 ng/μL, the concentration of the DNA polymerase P is 30 ng/μL, the concentration of the ssb protein is 30 ng/μL, and the concentration of the Exo Protein is 20 ng/μL.


SEQUENCE LISTING (13 sequences)

SEQ ID NO: 1 - DNA, 28 bp, Artificial Sequence
  Synthetic ASF-FP
cagatatagatgaacatgcgtctggaag

SEQ ID NO: 2 - DNA, 27 bp, Artificial Sequence
  Synthetic ASF-RP
atcctcatcaacaccgagattggcaca

SEQ ID NO: 3 - DNA, 43 bp, Artificial Sequence
  Synthetic ASF-P
  modified_base (27)..(27): I6FAMdT
  misc_feature (28)..(29): location of idSP
  modified_base (29)..(29): IBHQ1dT
tatctctgcgtggtgagtgggctgcatatggcgttaacaacat

SEQ ID NO: 4 - DNA, 362 bp, Artificial Sequence
  Synthetic P72
aggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttat

SEQ ID NO: 5 - DNA, 1092 bp, Deinococcus radiodurans
  misc_feature: Wild type RecA
atgagcaaggacgccaccaaagaaatctccgcccccaccgacgccaaggaacgcagcaaggccatcgaaacagccatgagccagatcgaaaaggccttcggcaagggctcgatcatgaaactgggcgccgagagcaaactcgacgtgcaggtcgtcagcaccggcagcctcagccttgacctcgcactgggcgtgggcggcattccgcgtggccgcatcaccgagatctacggccccgagtcgggcggcaagaccaccctggccctcgccatcgtcgcgcaggcccagaaagcgggcggcacctgtgcgtttatcgacgccgagcacgcgctcgacccggtgtacgcccgcgccctgggcgtgaacaccgacgaactgctggtgtcgcagcccgacaacggcgagcaggcgctcgaaatcatggaactgctggtgcgttcgggcgcgattgatgtggtggtcgtggactcggtggctgctctgaccccccgcgccgaaatcgagggcgacatgggcgacagcctgcccggtcttcaggcccgcctgatgtcgcaggcgctgcgcaagctgacggcgattctctccaagaccggcaccgccgccatcttcatcaaccaggttcgcgagaaaatcggcgtgatgtacggcaaccccgaaaccaccaccgggggccgggcgctgaagttctacgccagcgtgcgcctcgacgtgcgtaagatcggccagcccaccaaggtcggcaacgacgcggtcgccaacaccgtcaagatcaagaccgtgaagaacaaggtcgccgcccccttcaaggaagtcgaactcgcgctggtctacggcaagggcttcgaccagctcagcgacctcgtgggcctggccgccgacatggacatcatcaagaaggccggcagcttctactcctacggcgacgagcgcatcggccagggcaaggaaaagaccatcgcctacatcgccgagcgccccgagatggagcaggaaatccgcgaccgcgtgatggccgccatccgcgcgggcaacgcgggcgaagcaccggccctggcccccgcgcctgccgcgcccgaagccgccgaagcgtaa

SEQ ID NO: 6 - DNA, 1197 bp, Artificial Sequence
  Synthetic Novel typeRecA
atgagcaaggacgccaccaaagaaatctccgcccccaccgacgccaaggaacgcagcaaggccatcgaaacagccatgagccagatcgaaaaggccttcggcaagggctcgatcatgaaactgggcgccgagagcaaactcgacgtgcaggtcgtcagcaccggcagcctcagccttgacctcgcactgggcgtgggcggcattccgcgtggccgcatcaccgagatctacggccccgagtcgggcggcaagaccaccctggccctcgccatcgtcgcgcaggcccagaaagcgggcggcacctgtgcgtttatcgacgccgagcacgcgctcgacccggtgtacgcccgcgccctgggcgtgaacaccgacgaactgctggtgtcgcagcccgacaacggcgagcaggcgctcgaaatcatggaactgctggtgcgttcgggcgcgattgatgtggtggtcgtggactcggtggctgctctgaccccccgcgccgaaatcgagggcgacatgggcgacagcctgcccggtcttcaggcccgcctgatgtcgcaggcgctgcgcaagctgacggcgattctctccaagaccggcaccgccgccatcttcatcaaccaggttcgcgagaaaatcggcgtgatgtacggcaaccccgaaaccaccaccgggggccgggcgctgaagttctacgccagcgtgcgcctcgacgtgcgtaagatcggccagcccaccaaggtcggcaacgacgcggtcgccaacaccgtcaagatcaagaccgtgaagaacaaggtcgccgcccccttcaaggaagtcgaactcgcgctggtctacggcaagggcttcgaccagctcagcgacctcgtgggcctggccgccgacatggacatcaaaaatggctggtatgctcgtgaatttcttgacgaagaaactggcgagatgattcgcgaagaaaaatcttggcgtgcaaaagatactaactgcactacattctggatcaagaaggccggcagcttctactcctacggcgacgagcgcatcggccagggcaaggaaaagaccatcgcctacatcgccgagcgccccgagatggagcaggaaatccgcgaccgcgtgatggccgccatccgcgcgggcaacgcgggcgaagcaccggccctggcccccgcgcctgccgcgcccgaagccgccgaagcgtaa

SEQ ID NO: 7 - DNA, 27 bp, Artificial Sequence
  Synthetic ASF-FP1
tgtgccaatctcggtgttgatgaggat

SEQ ID NO: 8 - DNA, 26 bp, Artificial Sequence
  Synthetic ASF-RP1
ccacaccaacaataaccaccacgatg

SEQ ID NO: 9 - DNA, 45 bp, Artificial Sequence
  Synthetic ASF-P1
  modified_base (30)..(30): I6FAMdT
  misc_feature (31)..(32): location of idSP
  modified_base (33)..(33): IBHQ1dT
gttccaggtaggttttaatcctataaacataattcaatgggccat

SEQ ID NO: 10 - DNA, 29 bp, Artificial Sequence
  Synthetic ASF-FP2
ccgaacttgtgccaatctcggtgttgatg

SEQ ID NO: 11 - DNA, 29 bp, Artificial Sequence
  Synthetic ASF-RP2
aacgcaggtgacccacaccaacaataacc

SEQ ID NO: 12 - DNA, 45 bp, Artificial Sequence
  Synthetic ASF-P2
  modified_base (27)..(27): I6FAMdT
  misc_feature (28)..(29): location of idSP
  modified_base (30)..(30): IBHQ1dT
taggttttaatcctataaacatatattcatgggccatttaagagc

SEQ ID NO: 13 - DNA, 105 bp, Artificial Sequence
  Synthetic
aaaaatggctggtatgctcgtgaatttcttgacgaagaaactggcgagatgattcgcgaagaaaaatcttggcgtgcaaaagatactaactgcactacattctgg